(19) United States
(12) Patent Application Publication
(10) Pub. No.: US 2018/0014744 A1
(43) Pub. Date: Jan. 18, 2018

(54) MOTOR-ACTIVATED MULTI-FUNCTIONAL WRIST ORTHOTIC

(71) Applicant: Purdue Research Foundation, West Lafayette, IN (US)

(72) Inventors: Bradley Steven Duerstock, West Lafayette, IN (US); Shruthi Suresh, West Lafayette, IN (US); Sudhanshu Manda, West Lafayette, IN (US); Lucas Jacob, West Lafayette, IN (US); Daniel Madrinan Chiquito, West Lafayette, IN (US)

(73) Assignee: Purdue Research Foundation, West Lafayette, IN (US)

(21) Appl. No.: 15/703,914

(22) Filed: Sep. 13, 2017

Related U.S. Application Data
(60) Provisional application No. 62/362,011, filed on Jul. 13, 2016.

(51) Int. Cl. A61B 5/0488; A61F 2/72; A61F 2/58; A61F 5/01; A61F 2/70

(52) U.S. Cl. CPC A61B 5/04888; A61F 2/72; A61F 2/583; A61F 2002/701; A61F 5/01

(57) ABSTRACT

A multifunctional wrist orthotic comprising an electromyography (EMG) sensor having at least two electrodes for attachment to a wrist of a user, an intertial measurement sensor (IMU), a microcontroller unit (e.g., a Arduino® Mini) connected to the IMU, a power supply unit. The microcontroller unit is configured to perform two-tiered gesture recognition, with the first tier comprising a fine gesture sensed by the EMG sensor and the second tier comprising a gross gesture sensed by the IMU sensor.

### CROSS-REFERENCE TO RELATED APPLICATIONS

The present application is related to and claims the priority benefit of U.S. Provisional Patent Application Ser. No. 62/362,011, filed Jul. 13, 2016, the contents of which are hereby incorporated by reference in their entirety into this disclosure.

### TECHNICAL FIELD

The present application relates to orthotics, and more specifically, to a motor-activated wrist orthotic to assist Individuals with Cervical Spinal Cord Injuries with activities of daily living.

### BACKGROUND

There are approximately 12,500 new cases of Spinal cord injuries (SCI) every year in the United States alone. 53.9% of SCI are in the cervical region (C1-C7) and approximately 44% of these individuals have injuries in the C3-C6 region of the spinal cord (NSCISC, 2014). Daily manual activities such as unlocking doors with keys, holding utensils, writing, typing, using pointing devices, and swiping credit cards are extremely difficult for individuals with mid-cervical SCIs due to paralysis in the hand muscles preventing grasping and releasing and paralysis or weakness of wrist flexors and extensors. In order to stabilize a flaccid wrist, wrist orthoses or splints can be used to maintain the normal position of the hand and wrist. Wrist orthotics have often been used in rehabilitation of individuals with SCI to allow for the correct positioning of joints in the wrist, in order to maintain optimal muscle tone and structure. Tenodesis splints can be used for specific tasks such as assisting in picking up small objects by providing support to the thumb and forefinger. However, the limited motion of wrist braces for quadriplegics without the ability to flex or extend their wrists principally provides support. With the addition of a pocket in the palm strap, mid-cervical quadriplegics are able to insert dining utensils, pencils, pens, toothbrushes, or other tools to accomplish certain activities of daily living (ADL) independently.

For individuals with mid-level SCI (i.e. C4-05), common devices include surface Functional Electrical Stimulation (FES) systems in the form of a forearm sleeve which are applied during early rehabilitation to control voluntary wrist extension for grasping and flexion. Alternatively, several electromechanical exoskeletons have been constructed to provide basic support with hard metal hinges as manipulators. Most current systems assist individuals with SCIs through mechanical actuators or ratchet systems activated by existing functional muscles. The drawbacks of these devices are that they are bulky and cause fatigue to the individual. Common ways to control actuators on these systems include speech recognition and gesture recognition. Gesture recognition is often achieved through acceleration sensors or electromyography (EMG) signals. Unfortunately, EMG and accelerometer signals by themselves tend to be very noisy and can often lead to false positives. While improvements in speech recognition technology provide accurate control of actions during steady state, performance is significantly reduced in noisy environments. Therefore, improvements are needed in the field.

### SUMMARY

According to one aspect, the present disclosure provides a wearable multifunctional wrist orthotic (MFWO) which is activated by the user's limited motor function including EMG signals from the pronator teris (wrist muscle) and customized switch activation methods and concurrently performs distinct functions based on the recognition of different individualized gestures through an Inertial Measurement Unit (IMU). The microcontroller unit may be configured to perform two-tiered gesture recognition. The first tier comprises a fine gesture sensed by the EMG sensor and the second tier comprises a gross gesture sensed by the IMU sensor.

According to another aspect, a wrist orthotic is provided, comprising a rigid housing formed to fit around a portion of a forearm, wrist, and a portion of a hand of a user, a plurality of flexible straps for securing the housing to the user, an electromyography (EMG) sensor mounted to the housing and having at least two electrodes for attachment to the wrist of the user, an interial measurement unit (IMU) mounted to the housing, a microcontroller unit mounted to the housing and connected to the IMU, and a power supply unit mounted to the housing. The microcontroller may operate a device connected to the orthotic, such as an actuatable device, in response to recognized gestures.

### BRIEF DESCRIPTION OF THE DRAWINGS

In the following description and drawings, identical reference numerals have been used, where possible, to designate identical features that are common to the drawings.

FIG. 1 is a diagram showing a motor-activated wrist orthotic being worn by a user according to one embodiment.

FIG. 2 is a rigid housing for the wrist orthotic of FIG. 1 according to one embodiment.

FIG. 3 is a system diagram showing the control components of the orthotic of FIG. 1.

FIG. 4 is a diagram illustrating four gross gestures.

The attached drawings are for purposes of illustration and are not necessarily to scale

### DETAILED DESCRIPTION

In the following description, some aspects will be described in terms that would ordinarily be implemented as software programs. Those skilled in the art will readily recognize that the equivalent of such software can also be constructed in hardware, firmware, or micro-code. Because data-manipulation algorithms and systems are well known, the present description will be directed in particular to algorithms and systems forming part of, or cooperating more directly with, systems and methods described herein. Other aspects of such algorithms and systems, and hardware or software for producing and otherwise processing the signals involved therewith, not specifically shown or described herein, are selected from such systems, algorithms, components, and elements known in the art. Given the systems and methods as described herein, software not specifically shown, suggested, or described herein that is useful for implementation of any aspect is conventional and within the ordinary skill in such arts.

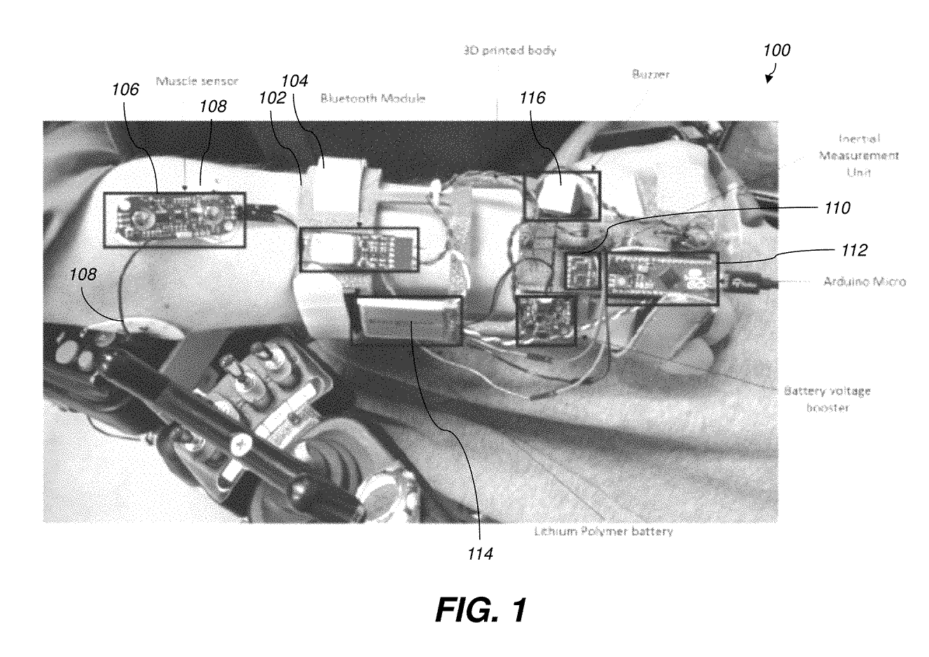

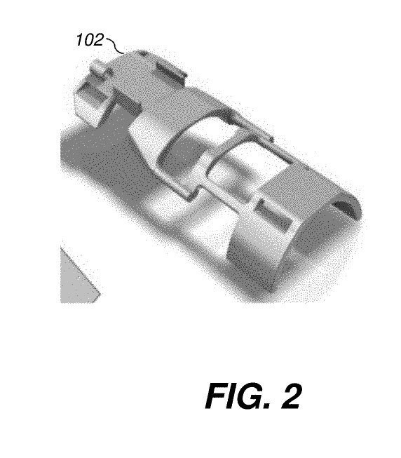

FIG. 1 shows a wrist orthotic 100 according to one embodiment. The wrist orthotic 100 includes an elongated rigid housing 102 (a further embodiment of the housing 102 shown in FIG. 2) formed to fit around a portion of a forearm and wrist of a user, and may optionally extend around a portion of the user's hand. The housing 102 may be formed from plastic or other suitable material to provide stability and support to the user's wrist. A plurality of flexible attachment straps 104 are attached to the housing 102 for securing the housing to the user as shown in FIG. 1. The orthotic 100 is designed to provide comfortable support and adheres to the design of current orthotics by including a wrap-around framework to support the sides of the hand to secure the correct positioning.

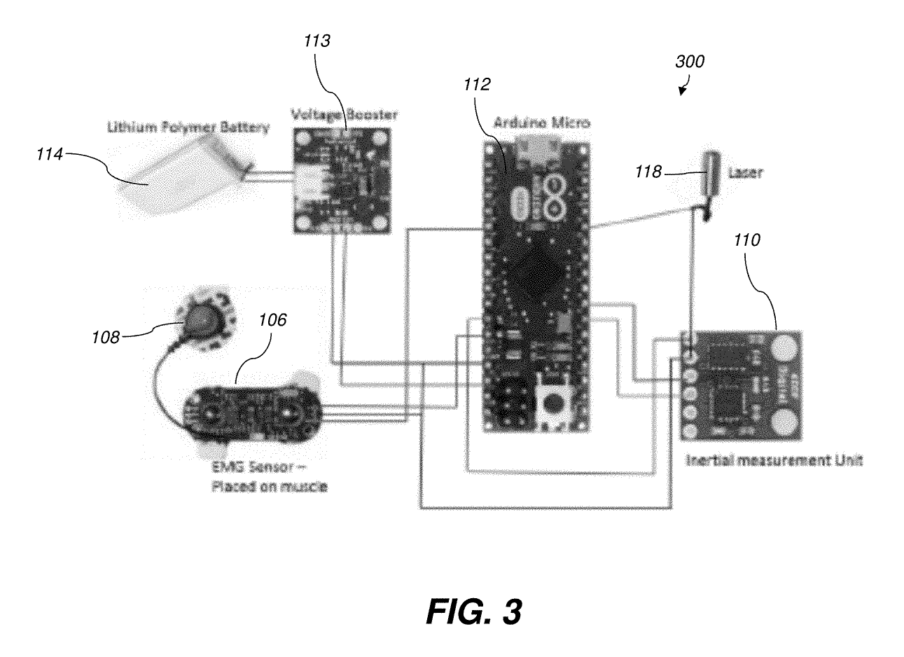

As shown in FIG. 1, and further in the system diagram 300 of FIG. 3, the wrist orthotic 100 includes an electromyography (EMG) sensor 106 mounted to the housing 102 and having electrodes 108 which attach to the surface of the user's arm and an intertial measurement unit (IMU) 110. The EMG sensor 106 and IMU 108 are connected to a microcontroller unit 112 (e.g., a Arduino® Mini) which receives output signals from the EMG sensor 106 and IMU 110 for processing and gesture recognition. Voltage booster 113 may be provided to increase the output voltage of the battery, sensors 106 114 or the microcontroller 112 as needed. The orthotic 100 may also include one or more actuators, such as a servo motor which manipulates an arm holding a key/card or a laser pointer. The sensors and microcontroller are powered by battery 114 (e.g., a recharable lithium ion or nicad battery). Buzzer 116 may be optionally included to provide auditory feedback to the user. Laser pointer 118 may also be optionally provided as shown and connected to the microcontroller 112. The orthotic 100 is designed to be lightweight (on the order of 300 grams), presenting an insignificant load to users and providing significant structural improvement over the commercially available options.

The wrist orthotic of FIG. 1 may be used to perform gesture recognition based on input from an EMG sensor 106 or touch-activated switch and IMU 110. To minimize the occurrence of false positives, a two-tier gesture recognition approach is implemented to control the system. The first tier is based on input received from the EMG sensor 106 or touch-activated sensor which detects a fine gesture allowing the activation of the second tier. The second tier is based on input from the IMU 110 which detects one of four or more gross gestures to perform the desired task as shown in Table 1 below.

TABLE 1

| Gesture | Motion |
|---|---|
| In-Out | Moving hand toward body (In) then |
| | away from body (Out) |
| Out-In | Moving hand away from body (Out) |
| | then toward body (In) |
| In-Hold | Moving hand toward body (In) and |
| | holding position (Hold) |
| Out-Hold | Moving hand away from body (Out) |
| | and holding position (Hold) |

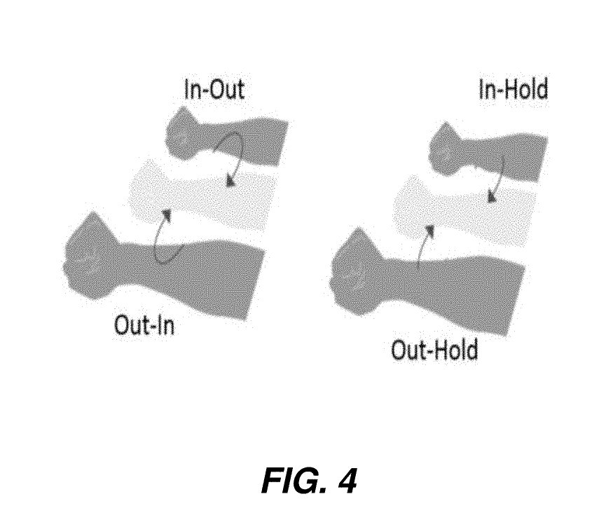

The EMG sensor 106 may comprise a light-weight sensor which measures action potentials from adhesive surface electrodes placed on top of the pronator teris (wrist muscle) of the user. The sensor 106 identifies a pattern of rapid supination-pronation of the wrist by the orthotic wearer, which then allows for appropriate activation of the IMU 110 during a preset time period. A touch-activated sensor may comprise a switch which is activated by contacting another surface in a specific position or providing close proximity to other body parts. To improve the accuracy of the gesture recognition, a dynamic time warping (DTW) based machine learning process is implemented by the microcontroller 112 in certain embodiments. The DTW process two time dependent sequences and identifies the similarities in them. In certain embodiments, after sensing a fine gesture from the EMG sensor 106, the IMU 110 recognizes four distinct gross gestures—In-Out, Out-In, In-Hold and Out-Hold, as shown in FIG. 4. These gestures were chosen for their comfort and ease of execution by individuals with cervical spinal cord injuries. Each of the gestures allows the control of one of the actuators (laser/servo) of the wrist orthotic. It shall be understood that more or less than four gross gestures may be recognized by the system 100.

The microcontroller 112, sensors 106 and 110, and other components recited herein may include one or more computer processors and memory which are communicatively connected and programmed to perform the data processing and control functionality recited herein. The program code includes computer program instructions that can be loaded into the processor, and that, when loaded into processor cause functions, acts, or operational steps of various aspects herein to be performed by the processor. Computer program code for carrying out operations for various aspects described herein can be written in any combination of one or more programming language(s), and can be loaded into memory for execution. The processors and memory may further be communicatively connected to external devices via a wired or wireless computer network for sending and receiving data.

The invention is inclusive of combinations of the aspects described herein. References to “a particular aspect” and the like refer to features that are present in at least one aspect of the invention. Separate references to “an aspect” (or “embodiment”) or “particular aspects” or the like do not necessarily refer to the same aspect or aspects; however, such aspects are not mutually exclusive, unless so indicated or as are readily apparent to one of skill in the art. The use of singular or plural in referring to “method” or “methods” and the like is not limiting. The word “or” is used in this disclosure in a non-exclusive sense, unless otherwise explicitly noted.

The invention has been described in detail with particular reference to certain preferred aspects thereof, but it will be understood that variations, combinations, and modifications can be effected by a person of ordinary skill in the art within the spirit and scope of the invention.

1. A wrist orthotic, comprising:
a rigid housing formed to fit around a portion of a forearm, wrist, and a portion of a hand of a user;
a plurality of flexible straps for securing the housing to the user;
an electromyography (EMG) sensor mounted to the housing and having at least two electrodes for attachment to the pronator muscle of a wrist of the user;
an interial measurement unit (IMU) mounted to the housing;
a microcontroller unit mounted to the housing and connected to the IMU; and
a power supply unit mounted to the housing.

2. The wrist orthotic of claim 1, wherein the power supply unit is a battery.

3. The wrist orthotic of claim 1, wherein the a first electrode is connected to the wrist near a pronator muscle of the user.

4. The wrist orthotic of claim 1, wherein the microcontroller unit is configured to perform two-tiered gesture recognition.

5. The wrist orthotic of claim 4, wherein the first tier comprises a fine gesture sensed by the EMG sensor due to supination-pronation of the user's wrist and the second tier comprises a gross gesture sensed by the IMU sensor.

6. The wrist orthotic of claim 5, wherein a gross gesture of the two-tiered gesture recognition includes a motion comprising moving a hand of the user toward the user body then away from the user body.

7. The wrist orthotic of claim 5, wherein a gross gesture of the two-tiered gesture recognition includes a motion comprising moving a hand of the user away from the user body then toward the user body.

8. The wrist orthotic of claim 5, wherein a gross gesture of the two-tiered gesture recognition includes a motion comprising moving a hand of the user toward the user body then holding the hand stationary for a predetermined minimum time period.

9. The wrist orthotic of claim 5, wherein a gross gesture of the two-tiered gesture recognition includes a motion comprising moving a hand of the user away from the user body then holding the hand stationary for a predetermined minimum time period.

10. The wrist orthotic of claim 1, further comprising an actuator mounted to the housing and configured to manipulate an arm for holding a key, wherein the microcontroller unit operates the actuator in response to a recognized gesture.

11. The wrist orthotic of claim 1, further comprising an actuator mounted to the housing and configured to manipulate an arm for holding a card, wherein the microcontroller unit operates the arm in response to a recognized gesture.

12. The wrist orthotic of claim 1, further comprising a laser pointer mounted to the housing, wherein the microcontroller unit operates the laser pointer in response to a recognized gesture.

13. A method, comprising:
a) using a processor, receiving a first input from an EMG sensor having at least two electrodes connected to skin of a user near a pronator teris muscle of the user;
b) upon receiving the first input, using the processor, activating an IMU mounted to the wrist of the user;
c) receiving a second input from the IMU, the second input representing a gross movement of the user's wrist; and
d) using the processor, based on the second input, determining a recognized gesture performed by the user; and
e) using the processor, sending a control signal to a device attached to the user's wrist to operate the device.

14. The method of claim 13, wherein the device is an actuator which operates a card holder.

15. The method of claim 13, wherein the device is a laser pointer.

16. The method of claim 13, wherein the device is a key holder.

17. The method of claim 13, wherein the gross gesture includes a motion comprising moving a hand of the user toward the user body then away from the user body.

18. The method of claim 13, wherein the gross gesture includes a motion comprising moving a hand of the user away from the user body then toward the user body.

19. The method of claim 13, wherein the gross gesture includes a motion comprising moving a hand of the user toward the user body then holding the hand stationary for a predetermined minimum time period.

20. The method of claim 13, wherein the gross gesture includes a motion comprising moving a hand of the user away from the user body then holding the hand stationary for a predetermined minimum time period.
